(19) United States
(12) Patent Application Publication
(10) Pub. No.: US 2025/0262401 A1
(43) Pub. Date: Aug. 21, 2025

(54) RESPIRATORY HUMIDIFICATION DEVICE AND METHOD

(71) Applicant: AUT Ventures Limited, Auckland (NZ)

(72) Inventors: Ahmed Kaleem Muez AL-ATTAR, Auckland (NZ); Ahmed AL-JUMAILY, Auckland (NZ)

(73) Assignee: AUT Ventures Limited, Auckland (NZ)

(21) Appl. No.: 19/057,735

(22) Filed: Feb. 19, 2025

(30) Foreign Application Priority Data
Feb. 20, 2024 (NZ) 808407

(51) Int. Cl. A61M 16/10

(52) U.S. Cl. CPC A61M 16/1045; A61M 2202/0007; A61M 2202/02; A61M 2205/0233; A61M 2205/12; A61M 2205/3368

(57) ABSTRACT

According to some embodiments, there is provided a respiratory humidifying device and method for humidifying inspiration airflow. The respiratory humidifying device (225) comprises a temperature-responsive moisture-exchanger material (260) configured to be in contact with the inspiration airflow and an expiration airflow; a CO2-responsive heat generating material (265) configured to be in contact with at least the expiration airflow; wherein the CO2-responsive heat generating material is in thermal communication with the temperature-responsive moisture-exchanger material.

### 1. CROSS-REFERENCE TO RELATED APPLICATION

This application claims priority to New Zealand Patent Application No. 808407, filed on 20 Feb. 2024, pending, which is incorporated herein by reference in its entirety.

### 2. TECHNICAL FIELD

The present disclosure relates to a device and method for humidifying inspired gases.

### 3. BACKGROUND

The heating and humidification of inspired air are achieved naturally by nasal conditioning. However, this cannot be accomplished during mechanical respiratory breathing due to the high pressure and ventilation air flow rate. Nevertheless, mechanical ventilation supports breathing during surgeries and is vital in airway pressure therapy and critical illnesses like asthma and Coronavirus infection. Thus, humidification of the inspired air is an essential process in mechanical ventilation to prevent inflammation in the respiratory system and chronic diseases like asthma, occlusion in the tracheobronchial structure, and damage in the lung or tracheal (Hyers et al., 2021).

Ordinary nasal breathing filters and protects against infiltrating particles and regulates the inspired air temperature and humidity to nearly optimal conditions to maintain the lungs' internal milieu. This transport process is mainly executed in the upper respiratory tract, where the air is evaporated using the hot water from the surfaces of the mucous membranes to achieve the isothermal saturation boundary (ISB) upon reaching the lungs. This boundary is optimally at body temperature (37° C.) and fully saturated with water vapor corresponding to 100% relative humidity (44 mg H2O/L of absolute humidity). The heat and moisture transfer process occurs due to a driving force created by the temperature and water concentration difference between ambient and humidity-inspired air and body temperature mucus membranes over a 160 cm2 area. This heat-exchanging mechanism is crucial and provides self-cleaning for the respiratory system under which mucociliary transport is at maximum capacity and the physical properties of the mucus layer are optimized (Gupta et al., 2014).

The nose's mucus traps particulate material and pathogens and moves towards the pharynx, then the upper respiratory tract; finally, the mucus is moved through the larynx and swallowed. The mucus covering the respiratory epithelium in the lungs is also conditioning the inspired air and trapping the particles to less extent than the nose, which is then moved and removed with the aim of coordinated ciliary activity. Any alteration in mucus rheology, which a range of pathophysiological conditions can cause, can affect the mechanical coupling of the cilia with the overlying mucus (Elad et al., 2008).

Whilst mechanical ventilation typically generates a heated and humidified inspiratory airflow for a patient, this airflow may lose moisture and/or heat as it progresses along conduits such as plastic tubing to the patient. A heat and moisture exchange (HME) device, also known as an artificial nose, may be used to supplement humidification and/or heating of inspired gases during the usage of anesthesia machines, ventilators, and positive airway pressure devices (including PAP, CPAP, APAP, and BiPAP). These devices preserve heat and moisture from a patient's exhaled humidified air during the exhalation, then recycle the heat and moisture to the next inspiratory stage to humidify the dehydrated air from the ventilator. Recent studies have demonstrated that patients demanding mechanical ventilation with sufficient hydration, without secretion issues, and with no record of harsh lung disease can handle these instruments for extended periods. Furthermore, the Studies indicate no increased chance of nosocomial infections with these passive humidification appliances, compared to ordinary hot bath heated humidifiers.

WO2022/203522 discloses an example HME in which a temperature-responsive polymer (TRP) is employed to absorb moisture from expired air and release the moisture into inspired air by controlling a heater dependent on the phase of the respiratory cycle. During the expiratory phase, the temperature of the TRP is controlled by the heater to be below a threshold temperature at which the TRP absorbs moisture from the surrounding air. The patient's exhaled airflow is passed over the TRP during this expiratory phase in order to absorb moisture from the patient's exhaled airflow or breath. During the inspiratory phase, the temperature of the TRP is controlled by the heater to be above a threshold temperature at which the TRP releases moisture into the surrounding air. The inspiration airflow is passed over the TRP during this inspiratory phase in order to absorb moisture from the TRP. This humidified inspiratory airflow is then directed to the patient. In this way, moisture from the patient's out-breath is recycled to assist with humidifying the patient's in-breath.

It is an object of the present invention to provide an improved device and method for humidifying inspired gases, or to overcome or ameliorate at least one disadvantage of the prior art. These objects or any other objects which may be referred to herein or taken from this specification are to be read disjunctively and with the alternative object of to at least provide the public with a useful choice.

In this specification where reference has been made to patent specifications, other external documents, or other sources of information, this is generally for the purpose of providing a context for discussing the features of the inventions disclosed herein. Unless specifically stated otherwise, reference to such external documents is not to be construed as an admission that such documents, or such sources of information, in any jurisdiction, are prior art, or form part of the common general knowledge in the art.

### 4. SUMMARY

In some examples, there is provided a respiratory humidifying device for humidifying inspiration airflow. The device comprises a temperature-responsive moisture-exchanger material configured to be in contact with the inspiration airflow and an expiration airflow and a CO2-responsive heat generating material configured to be in contact with at least the expiration airflow. The CO2-responsive heat generating material is in thermal communication with the temperature-responsive moisture-exchanger material.

This arrangement provides a simple passive mechanism for humidify inspiration airflow which may then be delivery to a patient in a medical setting or an individual requiring breathing assistance in other settings, such as using a CPAP machine or a scuba-diving breathing apparatus. Active control of the device is not required, and operation of the device is instead driven by a person's breathing cycle. Therefore electrical, heating and/or control of airflow are not required, resulting in a simpler and cheaper device.

In some examples, the CO2-responsive heat generating material may be configured to generate heat upon contact with the expiration airflow and to transfer at least some of the heat to the temperature-responsive moisture-exchanger material. The temperature-responsive moisture-exchanger material is configured to change temperate from below a critical solution temperature to above the critical solution temperature in response to the transfer of the heat from the CO2-responsive heat generating material. The temperature-responsive moisture-exchanger material is configured to release moisture above the critical solution temperature.

This allows the device to be optimised for different conditions by controlling the critical temperatures using different materials or different combinations of materials, as well as different amounts or densities of those materials. For example, a hospital setting may involve different ambient temperatures than a home or outside setting, leading to different temperature change requirements for the temperature-responsive moisture-exchanger material.

In some examples, the temperature-responsive moisture-exchanger material has a second critical solution temperature below which the temperature-responsive moisture-exchanger material is configured to absorb moisture. This allows for further optimisation options for different conditions.

In some examples, the CO2-responsive heat generating material is configured to be in contact with the inspiration airflow. This allows the inspiration airflow to be heated as well as humidified by the device.

In some examples, a by-pass arrangement is configured to direct only a part of the inspiration airflow to contact the CO2-responsive heat generating material. This allow control of the heating to be established, for example to be dependent on a characteristic of the inspiration airflow and/or the CO2-responsive heat generating material. Example characteristics include: a temperature of the inspiration airflow; a relative humidity of the inspiration airflow; a temperature of the CO2-responsive heat-generating material.

In some examples the bypass arrangement comprises a bi-metallic strip which is simple to implement, requiring no active control.

In some examples, the temperature-responsive moisture-exchanger material is configured to be in physical contact with the CO2-responsive heat generating material.

In other examples there may be an airgap. The airflows may be used to transfer heat between the materials.

In some examples, the respiratory humidifying device may be provided in a replaceable cartridge for a respiratory apparatus, such as may be used for medical patients. Such systems may comprise a ventilating apparatus to generate the inspiration airflow.

In some examples, there is provided a method of humidifying inspiration airflow. The method comprises directing an expiration airflow to contact a temperature-responsive moisture-exchanger material and a CO2-responsive heat generating material in thermal communication with the temperature-responsive moisture-exchanger material, and directing an inspiration airflow to contact the temperature-responsive moisture-exchanger material.

In some examples, the CO2-responsive heat generating material reacts with CO2 in the expiration airflow to generate heat to transfer to the temperature-responsive moisture-exchanger material, the temperature-responsive moisture-exchanger material increasing temperate from below a critical solution temperature to above the critical solution temperature in response to receiving the heat transferred from the CO2-responsive heat generating material, and the temperature-responsive moisture-exchanger material is configured to release moisture above the critical solution temperature.

Aspects of the inventions may also be said broadly to consist in the parts, elements and features referred to or indicated in the specification of application, individually or collectively, in any or all combinations of two or more of said parts, elements or features, and where specific integers are mentioned herein that have known equivalents in the art to which a said invention relates, such known equivalents are deemed to be incorporated herein as if individually set forth.

### 5. BRIEF DESCRIPTION OF THE DRAWINGS

The invention will now be described by way of example only and with reference to the drawings in which:

FIG. 1 shows a respiratory system for humidifying inspired gases, according to some embodiments;

FIG. 2 shows a respiratory humidifying device according to some embodiments;

FIG. 3 illustrates the molecular structure of the PNIPAM material of FIGS. 4 and 5;

FIG. 4 illustrates the chemical behavior of a Poly (N-isopropylacrylamide) polymer (PNIPAM)-Cotton fabric at surrounding temperatures below 32° C. when the PNIPAM material is in a hydrophilic state, according to some embodiments;

FIG. 5 illustrates the chemical behavior of a PNIPAM-Cotton fabric at surrounding temperatures above 32° C. when the PNIPAM material is in a hydrophobic state, according to some embodiments;

FIG. 6 shows a respiratory humidifying device according to some embodiments;

FIG. 7 shows respiratory airflow equipment for a respiratory system for humidifying inspired gases, according to some embodiments;

FIG. 8A to 8C illustrate operation of a respiratory humidifying device according to some embodiments over different phases of a respiratory cycle;

FIG. 9 is a flowchart illustrating operation of a respiratory humidifying device according to some embodiments;

FIG. 10 illustrates the chemical cycle of Soda-lime used in some embodiments;

FIG. 11 illustrates temperature of expiration airflow comprising CO2 with and without contact with Soda-lime;

FIG. 12 illustrates the measured temperature difference across three heat and moisture elements during operation of a respiratory humidifying device according to some embodiments; and

FIG. 13 shows a respiratory humidifying device according to some embodiments.

### 6. DETAILED DESCRIPTION OF THE INVENTION

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively. The term “about” as used herein means a reasonable amount of deviation of the modified term such that the end result is not significantly changed. For example, when applied to a value, the term should be construed as including a deviation of +/−5% of the value.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The terms “can” and “may” are used interchangeably in the present disclosure, and indicate that the referred to element, component, structure, function, functionality, objective, advantage, operation, step, process, apparatus, system, device, result, or clarification, has the ability to be used, included, or produced, or otherwise stand for the proposition indicated in the statement for which the term is used (or referred to) for a particular embodiment(s).

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

The term “about” as used herein means a reasonable amount of deviation of the modified term such that the end result is not significantly changed. For example, when applied to a value, the term should be construed as including a deviation of +/−5% of the value.

It is intended that reference to a range of numbers disclosed herein (for example, 1 to 10) also incorporates reference to all rational numbers within that range (for example, 1, 1.1, 2, 3, 3.9, 4, 5, 6, 6.5, 7, 8, 9 and 10) and also any range of rational numbers within that range (for example, 2 to 8, 1.5 to 5.5 and 3.1 to 4.7) and, therefore, all sub-ranges of all ranges expressly disclosed herein are hereby expressly disclosed. These are only examples of what is specifically intended and all possible combinations of numerical values between the lowest value and the highest value enumerated are to be considered to be expressly stated in this application in a similar manner.

Whenever a range is given in the specification, for example, a temperature range, a time range, or a composition range, all intermediate ranges and subranges, as well as all individual values included in the ranges given are intended to be included in the disclosure.

The following sets forth specific details, such as particular embodiments or examples for purposes of explanation and not limitation. It will be appreciated by one skilled in the art that other examples may be employed apart from these specific details. In some instances, detailed descriptions of well-known methods, nodes, interfaces, circuits, and devices are omitted so as not obscure the description with unnecessary detail. Those skilled in the art will appreciate that the functions described may be implemented in one or more nodes using hardware circuitry (e.g., analog and/or discrete logic gates interconnected to perform a specialized function, ASICs, PLAs, etc.) and/or using software programs and data in conjunction with one or more digital microprocessors or general purpose computers. Nodes that communicate using the air interface also have suitable radio communications circuitry. Moreover, where appropriate the technology can additionally be considered to be embodied entirely within any form of computer-readable memory, such as solid-state memory, magnetic disk, or optical disk containing an appropriate set of computer instructions that would cause a processor to carry out the techniques described herein.

Hardware implementation may include or encompass, without limitation, digital signal processor (DSP) hardware, a reduced instruction set processor, hardware (e.g., digital or analogue) circuitry including but not limited to application specific integrated circuit(s) (ASIC) and/or field programmable gate array(s) (FPGA(s)), and (where appropriate) state machines capable of performing such functions. Memory may be employed to storing temporary variables, holding and transfer of data between processes, non-volatile configuration settings, standard messaging formats and the like. Any suitable form of volatile memory and non-volatile storage may be employed including Random Access Memory (RAM) implemented as Metal Oxide Semiconductors (MOS) or Integrated Circuits (IC), and storage implemented as hard disk drives and flash memory.

Some or all of the described apparatus or functionality may be instantiated in cloud environments such as Docker, Kubenetes or Spark. This cloud functionality may be instantiated in the network edge, apparatus edge, in the local premises or on a remote server coupled via a network such as 4G or 5G. Alternatively, this functionality may be implemented in dedicated hardware.

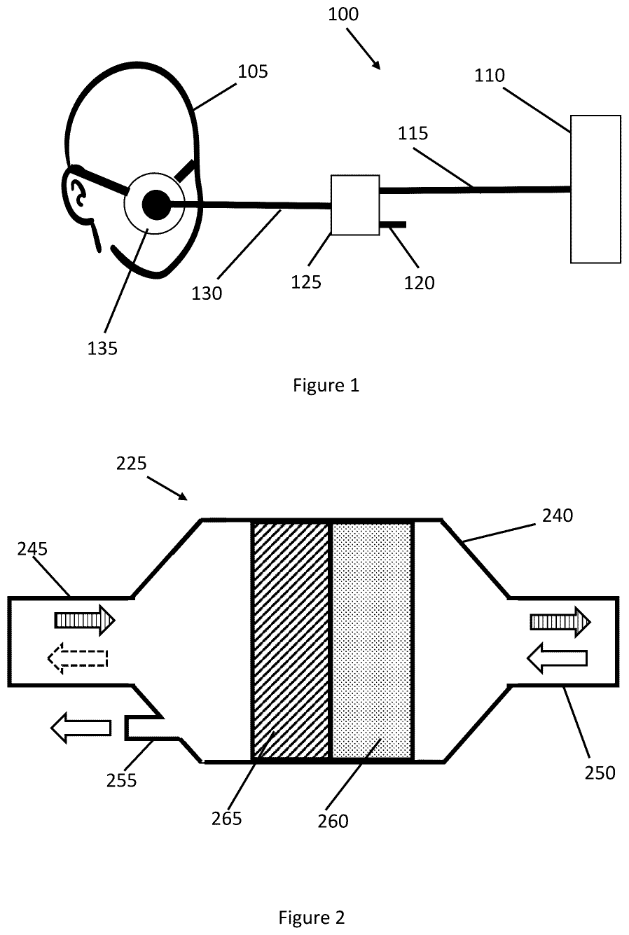

FIG. 1 illustrates a respiratory system for humidifying inspired gases, and which may be used for humidifying an inspiratory airflow to be provided to a patient. The respiratory system 100 comprises a ventilator apparatus 110, a respiratory humification device 125 and a patient interface 135 for interfacing with a patient 105.

The ventilator apparatus 110 is coupled to the respiratory humification device 125 using an inspiratory limb 115 to deliver inspiratory airflow or gases to the respiratory humification device 125. The inspiratory limb 115 may comprise plastic tubing or other conduits for directing the inspiratory airflow. The ventilator apparatus 110 may comprise a compressible air reservoir, turbine, pump or compressor to deliver high pressure air to the inspiratory limb 115. The ventilator apparatus 110 may comprise one or more gas sources such as compressed oxygen gas cylinders to mix with air from the compressible air reservoir. The ventilator may comprise a heater and humidifier to add heat and moisture to the inspiratory airflow. The ventilator may also comprise a controller to control when the inspiratory airflow is delivered to the inspiratory limb 115. Typically, this is controlled to synchronise with the patient's natural breathing cycle, so that the inspiratory airflow is started or increased when the patient is breathing in and prevented or reduced when the patient is breathing out.

The respiratory humification device 125 is also coupled to an expiratory limb 120 to deliver expiratory airflow or gases from the respiratory humification device 125. The expiratory limb 120 may simply vent the expiratory airflow to the external atmosphere, or in some embodiments this may be delivered back to the ventilator device 110 for venting or recycling. As with the inspiratory limb 115, the expiratory limb 120 may comprise plastic tubing or other conduits for directing the airflows. In some embodiments, the expiratory limb may simple be a valve from the respiratory humidification device 125 to an external atmosphere.

The respiratory humification device 125 is also coupled to a patient limb 130 to deliver the inspiratory airflow to the patient interface 105 and to receive the expiratory airflow from the patient interface 135. As with the inspiratory limb 115, the patient limb 130 may comprise plastic tubing or other conduits for directing the airflows.

The patient interface 135 interfaces with the patient 105 to exchange inspiratory and expiratory airflows through the patient's nose and/or mouth. The patient interface 135 may be a mask secured to the patient. In some embodiments, the respiratory humification device 125 may be incorporated into the patient interface 135, without the need for a separate patient limb 130.

FIG. 2 shows a respiratory humidifying device according to some embodiments. The respiratory humidifying device 225 comprises a housing 240 containing a temperature-responsive moisture-exchanger material 260 and a CO2-responsive heat generating material 265. The temperature-responsive moisture-exchanger material 260 and the CO2-responsive heat generating material 265 are in thermal communication with each other, for example they may be physically contacting each other or placed adjacent with a small airgap between them, sufficient for airflow to transfer heat between them. The housing 240 may also comprise particle filters.

In one embodiment, the temperature-responsive moisture-exchanger material 260 comprises a Poly (N-isopropylacrylamide) polymer (PNIPAM). This may be made of fibrous cotton fabric polymerized with the PNIPAM. The CO2-responsive heat generating material 265 comprises Sodalime, which may be implanted as particles in a fabric layer. However other materials may alternatively be used.

The housing 240 may be separated into two parts to access or replace one or both of the materials 260, 265. The housing comprises an inspiration port 245 to receive an inspiration airflow from a ventilator—shown as an arrow with shading. The housing comprises a common port 250 to deliver humidified inspiration airflow from the respiratory humidifying device 125 to a patient and to receive expiration airflow from a patient—shown as an arrow without shading. The housing may also comprise a separate expiration port 255 to vent the expiration airflow externally to atmospheric air. Alternatively, the expiration airflow may be directed to the inspiration port 245 for delivery back to the ventilator.

The expiratory airflow is directed to contact both the temperature-responsive moisture-exchanger material 260 and the CO2-responsive heat generating material 265. The temperature-responsive moisture-exchanger material 260 is configured to absorb moisture from the expiratory airflow. This occurs when the temperature of the temperature-responsive moisture-exchanger material 260 is below a critical solution temperature at which it becomes hydrophilic.

The CO2-responsive heat generating material 265 is configured to absorb CO2 from the expiratory airflow causing the generation of heat within the CO2-responsive heat generating material 265. As the CO2-responsive heat generating material 265 is in thermal communication with the temperature-responsive moisture-exchanger material 260, some of the generated heat transfers to the temperature-responsive moisture-exchanger material 260, causing the temperature of the temperature-responsive moisture-exchanger material to rise.

The inspiratory airflow is directed to contact both the CO2-responsive heat generating material 265 and the temperature-responsive moisture-exchanger material 260. In some embodiments described in more detail below, the inspiratory airflow may be directed only to contact the temperature-responsive moisture-exchanger material 260, or only part of the inspiratory airflow may be directed to contact the CO2-responsive heat generating material 265.

In contacting the CO2-responsive heat generating material 265, the inspiratory airflow may be heated due to the CO2-responsive heat generating material 265 having a temperature above the temperature of the inspiratory airflow. This may be caused by the previously described heating due to absorbing CO2 from the expiratory airflow.

In contacting the temperature-responsive moisture-exchanger material 260, the inspiratory airflow absorbs moisture released from the temperature-responsive moisture-exchanger material 260. This occurs when the temperature of the temperature-responsive moisture-exchanger material 260 is above the critical solution temperature at which it becomes hydrophobic.

In an embodiment, the critical solution temperature is 32 C. Depending on the material used, the critical solution temperature may be an upper critical solution temperature (UCST) when the phase transition (from hydrophobic and hydrophilic, and vice versa) occurs below the UCST, and may be a lower critical solution temperature (LCST) when the phase transition occurs above the LCST.

The temperature of the temperature-responsive moisture-exchanger material 260 is increased by heat transfer from the CO2-responsive heat generating material 265 and is reduced when that heat transfer stops. The heat transfer occurs as a result of exhalation by the patient during which CO2 is absorbed by the CO2-responsive heat generating material 265 from the expiratory airflow. The heat may be transferred by direct conduction between the materials and/or with airflow passing between them. With sufficient heat transfer, the temperature of the temperature-responsive moisture-exchanger material 260 rises above the critical solution temperature changing the material from a hydrophilic state (absorbing moisture from the expiratory airflow) to a hydrophobic state (releasing moisture into the inspiratory airflow).

When the patient's exhalation finishes, inspiration airflow is introduced into the respiratory humidifying device 225 which absorbs the moisture released by the temperature-responsive moisture-exchanger material 260 and may additionally absorb some of the heat generated by the CO2-responsive heat generating material 265. As the patient inspiration continues, the heat transfer reduces and the temperature of the temperature-responsive moisture-exchanger material 260 falls, due to normal thermodynamic heat transfer to the surrounding environment and/or heat transfer to the patient via heated inspiratory airflow. This causes the temperature of the temperature-responsive moisture-exchanger material 260 to fall below the critical solution temperature changing the material from the hydrophobic state (releasing moisture) into the hydrophilic state (absorbing moisture).

With suitable configuration of the temperature-responsive moisture-exchanger material 260 and the CO2-responsive heat generating material 265, the changes between hydrophilic and hydrophobic states of the temperature-responsive moisture-exchanger material 260 coincides with the flow of expiratory and inspiratory airflow respectively. This enables with respiratory humidifying device 225 to passively humidify the inspiratory airflow to a patient simply by utilising the normal expiratory and inspiratory airflows and without the need for separate control of the heating of the temperature-responsive moisture-exchanger material 260. This simplifies the respiratory humidifying device 225 and means that no electrical supply wires are required to be fed into the respiratory humidifying device 225, which simplifies the respiratory system 100, lowers costs, and reduces the number of external components to be coupled to the patient. In some alternative embodiments, the passive humidifying function of the respiratory humidifying device 225 may be supplemented by external heating (and/or cooling) of the inspiratory airflow and/or the temperature-responsive moisture-exchanger material 260.

In an embodiment, the temperature-responsive moisture exchanger material 260 is made of materials such as Poly n-isopropylacrylamide, which can respond to a change in temperature and are used for biomedical applications, including drug delivery, tissue engineering, and gene delivery. Temperature-responsive polymers exhibit a volume phase transition at a specific temperature, which causes a sudden change in the solvation state. Polymers become hydrophobic upon heating and have a so-called Lower Critical Solution Temperature (LCST).

Typical LCST polymers include N-isopropylacrylamide (NIPAM), N-diethylacrylamide (DEAM), methylvinylether (MVE), and N-vinylcaprolactam (NVCI) as monomers. Typical UCST materials may be based on a combination of acrylamide (AAm) and acrylic acid (AAc) and its corresponding co-polymers. Poly(N-alkylacrylamide) such as poly(N-isopropylacrylamide) (PNIPAM) or poly(N,Ndiethylacrylamide) (PDEAM).

PNIPAM is a good candidate as a thermos-responsive polymer, even though a second polymer in this class has a nearly identical transition temperature: PDEAM. However, the transition temperature of PDEAM depends on the tacticity of the polymer, which is in contrast to PNIPAM. Its biocompatibility and the position of the LCST at 32-33° C. make PNIPAM an absorbing material, e.g., for controlled release application. The LCST of PNIPAM is independent of the molecular weight and the concentration, but it can be changed upon shifting the hydrophilic/hydrophobic balance.

A high precision balance may be maintained between the different materials and equipment used to synthesize the PNIPAM polymer. For example, the materials and equipment used to synthesize the PNI PAM-Cotton material are the following: commercial cotton fabric, commercial soap, distilled water, ethanol (99.5%), methanol (99.9%), dry tetrahydrofuran (TH F) (99.8%), 4-(dimethylamino) pyridine (DMAP) (99%), triethylamine (TEA) (99%), bromoisobutyrate (BiB) (99%), copper(I)bromide (CuBr) (98%), N-Isopropylacrylamide (NIPAM) (99.5%), pentamethyldiethylenetriamine (PM DETA) (99%), ethyl 2-(bromomethyl) acrylate (EBMA) (98%), dimethylformamide (DM F) (99.8%), deuterated 4-methanol (Methanol-d4) (99.96%) and sodium hydroxide (NaOH) (97%), nitrogen flow, vacuum/Incubator chamber, with a high precision balance between the synthesized materials.

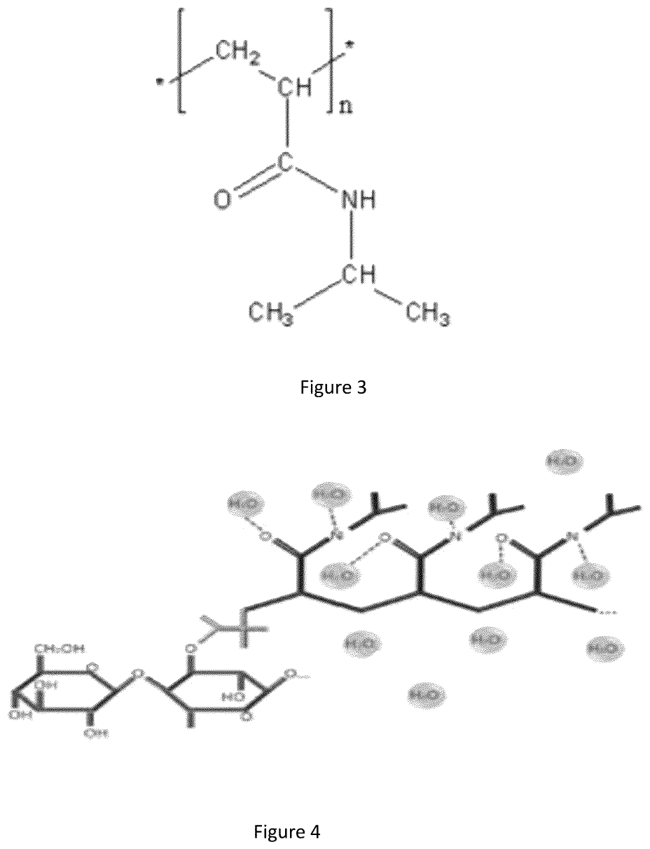

PNIPAM is an eco-friendly temperature-sensitive polymer that undergoes a coil-to-globule transition (hydrophilic-to-hydrophobic) in an aqueous solution at a specific temperature of 32° C. called lower critical solution temperature (LCST). Poly n-isopropylacrylamide may be abbreviated as PNI PAM, PNI PAm, pNI PAm, PNIPA, pNIPA, PNIPAAm, pNIPAAm, PNI PAA, pNIPAA. The molecular structure of PNIPAM is illustrated in FIG. 3.

Below the polymer's lower critical solution temperature (LCST) and in the presence of water, the PNIPAM molecules are predominantly hydrophilic. The amide groups form intermolecular hydrogen bonds with the surrounding water molecules. Above the lower critical solution temperature, the hydrophobic isopropyl-methyl groups tend to establish polymer-polymer interactions, which are energetically more favorable at higher temperatures. A change from coil-to-globule configuration accompanies this transition.

Other suitable polymerizes could be used Poly(N-vinyl caprolactam) (PVC) which has a transition temperature within the settings of these applications of 33° C.

Poly(methyl vinyl ether) (PMVE) Poly(methyl vinyl ether) has a transition temperature of precisely 37° C. The polymer exhibits a typical type III de-mixing behavior, contrasting the thermal behavior of PNI PAM. PMVE has to be synthesized by cationic polymerization using inert conditions. Nucleophiles like alcohol or amino groups cannot be tolerated during the synthesis, which limits the potential of PMVE Poly(acrylic acid-co-acrylamide) such as poly acrylic acid (PAAc) or polyacrylamide (PAAm). An interpenetrating network of Poly(acrylicacid) and polyacrylamide is one of the few examples of a system with UCST behavior within the biomedical setting. The transition temperature is at 25° C.

The UCST behavior is caused by the cooperative results reaching from the hydrogen bonding between AAc and AAm units. Elastin-like oligo- and polypeptides such as Poly(GVGVP) Poly(pentapeptide) of elastin (G: Glycine, V: Valine, and P: Proline). Polypeptides can also show LCST behavior when well-balanced hydrophilic and hydrophobic residues. A polymer made out of the pentapeptide GVGVP as a repeating unit exhibits a volume phase transition at 30° C., the hydrophobic folding and assembling transition.

Below the phase transition, water molecules are structured around the polymer molecule; the attractive forces weaken upon heating and finally go into the bulk phase. The temperature-responsive polymer (with its specific LCST) and the substrate material should be selected according to the application, the specific design, and the practical environmental conditions such as temperature and humidity. The LCST of a specific thermo-sensible polymer can be slightly modified during the polymerization process by varying the grafting density or the chain length.

This embodiment is designed for an ambient temperature ranging from about 20° C. to 25° C. and around 50% relative humidity. The thermo-sensible polymer and its LCST are chosen to be above the room temperature to avoid a cooling system in the design, PNI PAM, with LCST at 32° C. For similar practical conditions, LCST is preferably above room temperature, between 25° C. and 32° C., more preferably the LCST is between 28° C. and 32° C. even more preferably the LCST is between 30 degrees Celsius and 34 degrees Celsius, and most preferably the LCST is about 32° C.

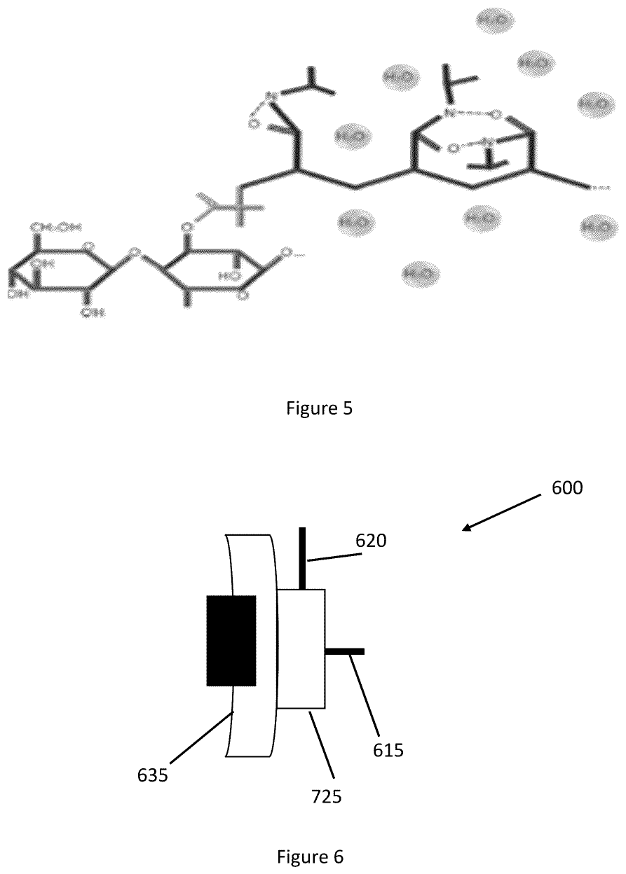

Referring to FIG. 4 and FIG. 5, the absorption and desorption process is outlined. Below 32° C. (lower critical solution temperature), as shown in FIG. 4, the polymer has a coil structure and hydrophilic state and forms intermolecular hydrogen bonds between oxygen, nitrogen, and the surrounding water molecules. Above lower critical solution temperature, as shown in FIG. 5 the polymer has a globule structure and hydrophobic state, and establishes polymer-polymer interactions between oxygen and nitrogen, and releases previously absorbed water molecules.

The structural changes of a temperature-responsive polymer (PNIPAM) leads to reversible and repeatable switching between two extreme wettability states, superhydrophobic and superhydrophilic. This may be enhanced by combining this material with a highly rough surface of a fibrous material For example a PNIPAM-Cotton fabric can absorb water from a humid environment below the lower critical solution temperature and release it upon a temperature change at constant humid airflow.

In an embodiment, the temperature-responsive polymer layer may be manufactured using a piece of cotton fabric polymerized by PNIPAM (PNIPAM Cotton fabric). However, other fibrous materials such as natural fibers (Cotton, Linen, Chitin, or Chitosan), synthetic or processed fibers (Rayon, Polyvinyl alcohol (PVA), or Polypropylene (PP) between others), or a mix could be used.

Commercially available cotton fabric is used as substrate. Cotton fiber is typically composed of approximately 90% of cellulose (C6H10O5)n, which is an organic polysaccharide made of a linear chain of several b(1->4) linked D-glucose units. Example cotton dimensions that could be used are three mm in height, five cm in width, and five cm in depth. The hydrophilic surface of the cotton fabric may be coated with the PNIPAM polymer coating utilizing an atom transfer radical polymerization (ATRP) process. With this method, it is possible to graft a small percentage of PNIPAM brushes directly from the cotton fabric surface. The bare-cotton fabric becomes fully covered with a thick and rough PNIPAM layer, which increases the diameter of the fibers, and the polymer chains grow from the fiber's surface with a concentric orientation creating a highly rough surface at the micrometer level.

CO2-responsive heat-generating material 265 may be made of materials such as traditional Soda-lime; alternatively, pellet mixtures of medically graded CO2-reacting products such as Sodasorb or Spherasorb formulas may be employed.

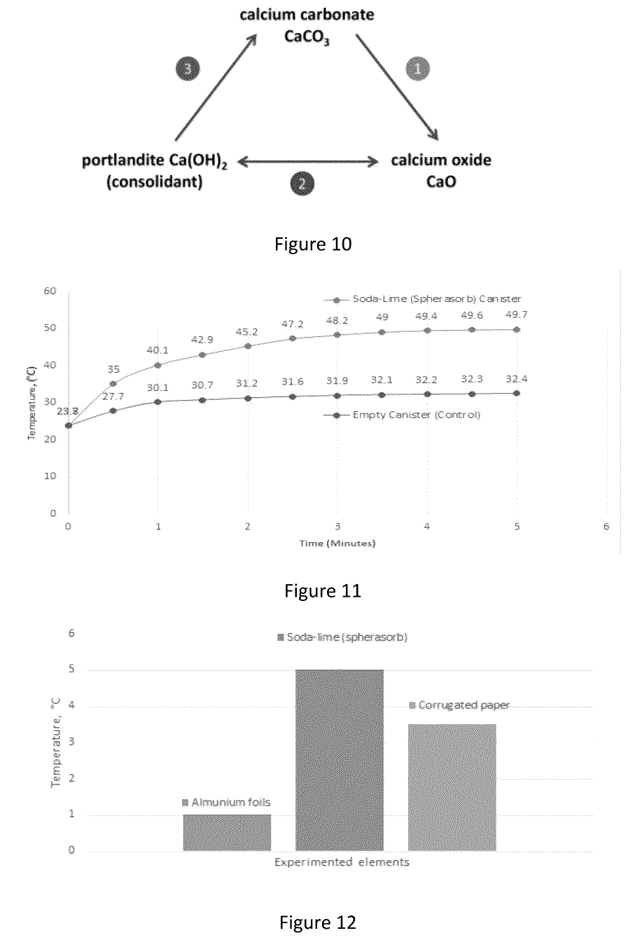

In an embodiment, the CO2-responsive heat generating material may utilize exothermic chemical reactions of a medically safe and graded Soda-lime to generate heat using carbon dioxide (CO2) gas from the exhaled air as fuel for heat generation. Soda-lime uses three main connected chemical reactions, as shown in FIG. 10.

Reaction One: Decarbonisation, which is the thermal decomposition of calcium carbonate:

CaCO3+heat→CaO+CO2

Reaction two: Hydration of Calcium oxide,

CaO+H2O→Ca(OH)2

or Dehydration of Calcium hydroxide,

Ca(OH)2+heat→CaO+H2O

Reaction Three: Carbonation of Calcium hydroxide with atmospheric CO2 gas

Ca(OH)2+CO2→CaCO3+H2O

The main compound consumed in the soda-lime is the Calcium hydroxide Ca(OH)2 of about 95%. The chemical composite of soda-lime crystals may vary depending on the manufacture, but it is typically formulated in the following components:
Calcium hydroxide (Ca(OH)2): 80%, and some manufacturers may quote up to 97%
Bound water (H2O): 15% and some manufacturers may quote up to 14-20%
Sodium hydroxide (NaOH): 4%, and some manufacturers may reduce it to 2%
Potassium hydroxide (KOH): less than 1% or null
Zeolite: Up to 5% or null
Silica (SiO₂): 0.2%

Soda-lime may contain several accelerators or catalytic, which may assist in changing the characteristics of the Soda-lime; for example Sodium hydroxide (NaOH). Alternatively, Calcium hydroxide Ca(OH)2 or potassium hydroxide KOH may be utilized as they are also medically graded options. Silica (SiO2) may also be added to produce calcium and sodium silicate, which hardens the granules that otherwise disintegrate into powder in trace amounts. The chemical process of different Soda-lime CO2 Absorptions is an exothermic chemical reaction between a proprietary mixture of calcium hydroxide, sodium hydroxide, and water.

Soda-lime CO2 absorbent may also contain Colour indicators dye that change the colour of soda-lime as it is exhausted by progressive absorption of CO2. There are different compounds and colours like Phenolphthalein (red to white), Ethyl violet (white to purple), Clayton (yellow-pink to cream), Titan (yellow-pink to cream), and Mimosa (red to white). The rate of CO2 absorption from exhalation depends on the capacity of the canister, FGF rate, and rate of CO2 production. CO2 absorbents may be changed whenever the Colour change indicates exhalation of the Soda-lime.

Sodium hydroxide (NaOH) is a catalyst for the reaction as it is reformed in the final stage. The compound consumed in the Soda-lime is Ca(OH)2. Some CO2 may react directly with Ca(OH)2 but in a much slower reaction. Efficient CO2 absorption by soda-lime requires water, which can be obtained from the CO2 absorbent, moisture from exhaled air, or the chemical reaction between CO2 and Ca(OH)2.

This exothermic reaction is zonal, causing the heat produced to be uneven throughout the canister. The amount of heat produced by soda-lime will depend on the quantity of absorbed CO2, ambient temperature, CO2 gas flow rate, and the thermal property of the canister where the chemical reaction is taking place. The chemical reaction of carbon dioxide with a strong base like Soda-lime is exothermic, in which heat and water are produced due to the dissolution of the Alkali salts as calcium hydroxide Ca(OH)2 and, to a lesser extent, Sodium hydroxide (NaOH) in water.

A high temperature can be generated from the exothermic reaction that may reach more than 60° C. in the center of a soda-lime layer and five degrees only temperature difference across the HME elements during operation with the temperature at edge of the CO2 absorption canister above the PNIPAM UCST (i.e., 39° C.). Some researchers demonstrated that in the face mask of an apparatus, the temperature range was from 39° C. to 42° C. Nevertheless, the heat generated by the exothermic reply will mainly depend on the properties of the Soda-lime crystals and the manufacturing formula.

In an embodiment, once a fabric is created, a PNIPAM-cotton layer is attached to a Soda-lime layer. The PNIPAM-Cotton layer may achieve a water vapor release rate of 24.2±1.054%/min (mean±standard deviation, n=3), corresponding to the weight percentage of water vapor released per minute at an LCST of 32° C. This means that below 32° C. the fabric absorbs the surrounding water vapor molecules and releases them when the temperature is higher than 32° C. achieving a water vapor release rate up to 24.2±1.054%/min (mean±standard deviation, n=3).

FIG. 6 illustrates a respiratory system according to some embodiments. The respiratory system 600 comprises a respiratory humidifying device 625 mounted to or within a person interface 635 such as a mask with a securing strap (shown in black). The respiratory humidifying device 625 may be configured as previously described, for example with respect to FIG. 2, and may comprise or be coupled with an inspiratory inlet or limb 615 and may also comprise with an expiratory outlet or limb 620. One or more of the limbs 615, 620 may be coupled to an external apparatus such as a ventilator or other breathing equipment such as scuba-diving equipment. Alternatively or additionally, one or more of the limbs 615, 620 may simply comprise an orifice to the external atmosphere. This may include a one-way valve to prevent airflow being drawn into the expiratory limb 620 or to prevent airflow from exiting the inspiratory limb 615.

A simple arrangement with inspiratory and expiratory orifices to/from atmosphere may be employed in applications where a person may benefit from humidifying the external air but does not need medical intervention. For example, a person with asthma may utilize a mask with an integral respiratory humidifying device on an “as needed” basis. Such arrangements may allow replacement of the internal layers as needed or they may be made as single use disposable units, for example available to airline passengers during a flight.

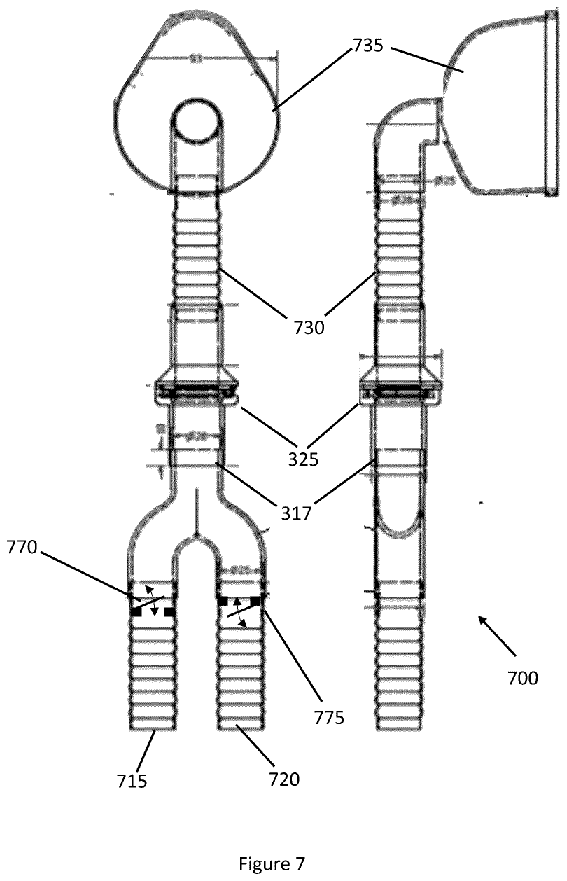

FIG. 7 shows respiratory airflow equipment for a respiratory system for humidifying inspired gases, according to some embodiments. The respiratory airflow equipment 700 comprises a patient interface 735 such as a mask coupled to a patient limb 730 such as flexible plastic tubing which is coupled to a cartridge holder 735. The cartridge holder 735 is also coupled to a ventilator limb 717 which splits into an inspiratory limb 715 and an expiratory limb 720. The inspiratory limb 715 receives an inspiratory airflow from a ventilator and delivers this to the cartridge holder 735. The expiratory limb 720 receives an expiratory airflow from the cartridge holder 735 and delivers this to the ventilator.

Each of the inspiratory and the expiratory limbs 715, 720 comprise a one way valve 770, 775 to ensure airflow in only one direction. The inspiratory limb 715 comprises a valve 770 to allow inspiratory airflow from the ventilator to the cartridge holder 725 but prevent expiratory airflow from the cartridge holder 725 to the ventilator. The expiratory limb 720 comprises a valve 775 to allow expiratory airflow from the cartridge holder 725 to the ventilator but prevent inspiratory airflow from the ventilator to the cartridge holder 725.

The cartridge holder 725 is configured to receive a respiratory humidifying device, such as the device 225 of FIG. 2. In this configuration, the expiratory airflow may be configured to exit through the inspiratory port 245. As the respiratory humidifying devices of some embodiments are passive and do not require wires or other connecting parts, they may be easily inserted into and removed from the cartridge holder 725. The respiratory humidifying devices may then be replaced as required, for example between patients or when the supply of CO2 responsive heat generating material is exhausted.

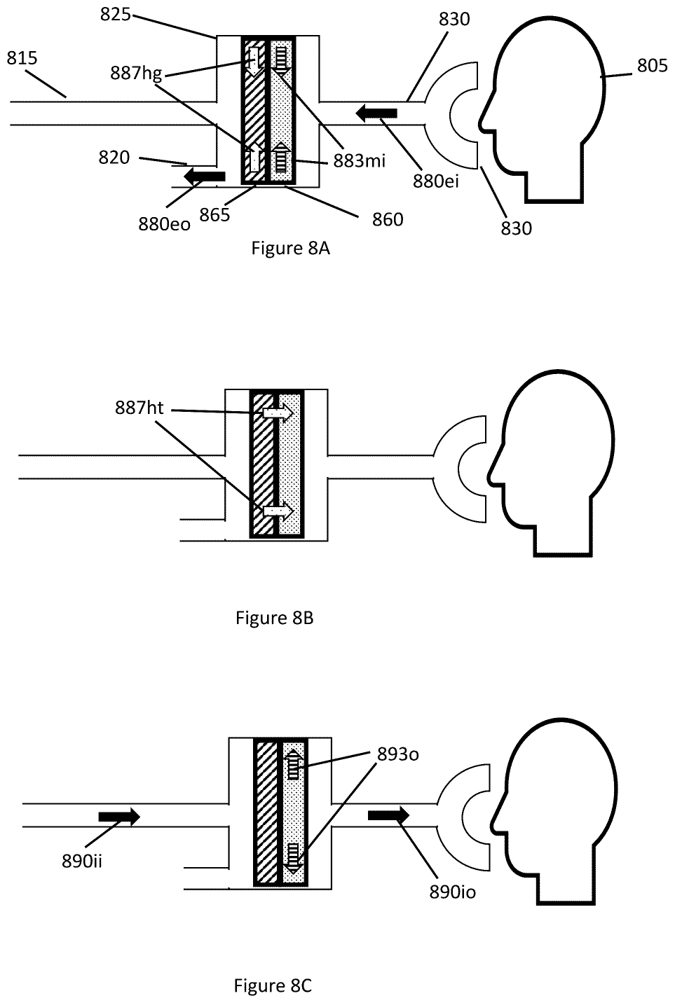

FIGS. 8A-8C illustrates operation of a respiratory humidifying device 825 according to some embodiments over different phases of a respiratory cycle. The respiratory humidifying device 825 may be configured as described for the device 225 of FIG. 2. FIG. 8A illustrates expiration at room temperature, typically below 32° C., the polymer lower critical solution temperature (LCST) of PNIPAM 860. Under these conditions, the PNIPAM-molecules are predominantly hydrophilic, and the amide groups form intermolecular hydrogen bonds with the surrounding water molecules. Expiration airflow is generally at 100% relative humidity, and the PNIPAM-Cotton material becomes moisture saturated and absorbs the maximum number of water molecules. This absorption is illustrated by the arrows 883mi pointing into the temperature-responsive humidity absorbing material 865.

The CO2-responsive heat generating material 865, for example Sodalime, takes the CO2 gas from exhaled air, and produces heat from an exothermic reaction. Levels of CO2 are typically high in exhaled airflow, when compared with the ambient atmosphere. The layer of material 860 retains the heat inside the respiratory humidifying device 825. This generation of heat is illustrated by the arrows 887hg pointing into the CO2-responsive heat generating material 865.

Heat from the CO2-responsive heat generating material 865 is transferred to the temperature-responsive moisture exchanger material 860. Depending on the configuration of the respiratory humidifying device 825, heat may be conducted soon after the CO2-responsive heat generating material 865 begins heating. This heat transfer may accelerate (or be started) when the expiration flow has ended (after approximately three seconds), as illustrated in FIG. 8B with arrows 887ht. The beginning of inhalation may also transfer heat from the CO2-responsive heat generating material 865 to the temperature-responsive moisture exchanger material 860 in the inspiratory airflow itself.

The heat transfer from the CO2-responsive heat generating material 865 to the temperature-responsive moisture exchanger material 860 raises the temperature above the UCST (39° C. for PNIPAM). Under these conditions, the hydrophobic isopropyl-methyl groups tend to establish polymer-polymer interactions, and the water molecules previously absorbed are released into the inspiration airflow. This is illustrated in FIG. 8C by outwardly pointing arrows 8930.

In other words, the heat-responsive moisture exchanger material (e.g., PNIPAM) has both features, a UCST for transitioning from hydrophobic to hydrophilic and an LCST for transitioning from hydrophilic to hydrophobic, and which are different from each other. Therefore, when the ambient temperature surrounding the material rises above the UCST, the material transitions from hydrophobic to hydrophilic and when the temperature falls below the LCST, the material transitions from hydrophilic to hydrophobic.

The inspiratory airflow may have originally been at a lower humidity and temperature, but after passing through the respiratory humidifying device, the humidity has been increased. The temperature of the inspiratory airflow may also have been increased by directly contacting the heated CO2-responsive heat generating material 860 and the heated temperature-responsive moisture exchanger material 865. The inspiratory airflow would typically have low levels of CO2 and so would not cause the CO2-responsive heat generating material 860 to heat at this phase.

The humidified and heated inspiratory airflow then moves towards the patient which reduces the temperature of the temperature-responsive moisture exchanger material 860. When the patient again begins exhaling air, this may also cool the temperature-responsive moisture exchanger material 860 as heat from this material is transferred downstream to the CO2-responsive heat generating material and out of the respiratory humidifying device 825 in the expiratory airflow. When the temperature of the temperature-responsive moisture exchanger material 860 falls below the LCST (32 C for PNIPAM), the temperature-responsive moisture exchanger material 860 will again begin absorbing moisture from the expiratory airflow.

The process will then continue during the mechanical ventilation providing the patient with warm and humidified breathing air.

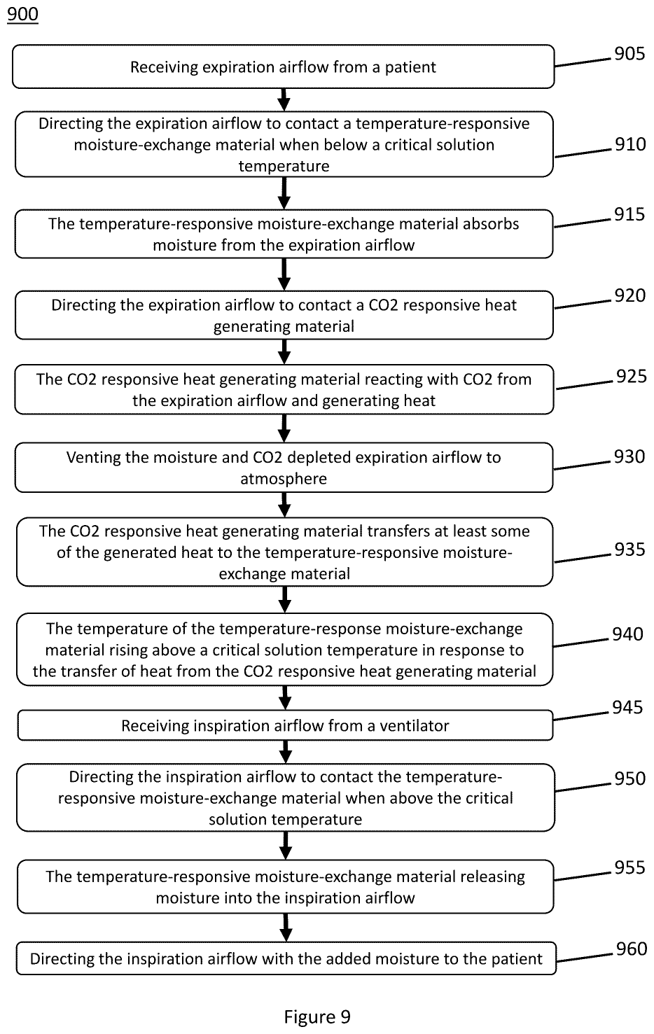

FIG. 9 is a flowchart illustrating operation of a respiratory humidifying device according to some embodiments. The respiratory humidifying device may be similar to those described and illustrated in FIG. 1, 2 or 4, 7-8c and 13, however other arrangements may be operated with this method.

The method of operation 900 of a respiratory humidifying device receives expiration airflow from a patient at 905. This corresponds with a patient breathing out into an interface which is coupled by a flow path to the device. The patient's breath will typically be warmer than the surrounding ambient air, will be largely saturated with moisture or water droplets (high relative humidity), and will comprise a much larger proportion of CO2 than the surrounding ambient air.

At 910, the method directs the expiration airflow to contact a temperature-responsive moisture exchanger material such as PNIPAM, when this is below its critical solution temperature. Below this temperature the material is hydrophilic and begins absorbing moisture from the expiration airflow at 915. As the patient continues to breath out, more moisture is absorbed by the temperature-responsive moisture exchanger material. These steps may be implemented passively by suitable configuration of an airflow pathway from an inlet port receiving the expiration airflow to the temperature-responsive moisture exchanger material. Alternatively, an active pathway may be used, for example by employing valves controlled to allow the expiration airflow to contact the material only during prescribed time periods. These time periods may be synchronised with the patient's breathing cycle.

At 920, the method directs the expiration airflow to contact a CO2 responsive heat generating material such as Sodalime. The expiration airflow will typically have passed proximate to the temperature-responsive moisture exchanger and so may be depleted of some moisture. However, alternative arrangements are possible in which separate airflow pathways may be employed to direct separate portions of the expiration airflow to the temperature-responsive moisture exchanger material and the CO2 responsive heat generating material respectively. In response to reacting with the CO2 in the expiration airflow, the CO2 responsive heat generating material begins generating heat at 925. This causes the material to increase in temperature and depletes the expiration airflow of some CO2.

At 930, the moisture and CO2 depleted expiration airflow is vented to the surrounding atmosphere. This may be implemented using a simple one-way valve. Alternatively, the moisture and CO2 depleted expiration airflow may be retained within a respiration system for reconditioning and use in providing an inspiratory airflow for the patient.

At 935, some of the heat generated by the CO2 responsive heat generating material is transferred to the temperature-sensitive moisture-exchange material. This may be achieved using conduction when the two materials are in physical contact. Additionally or alternatively, convection using another airflow such as an inspiration airflow may be employed in which this airflow is first directed to contact the CO2 responsive heat generating material and then to contact the temperature-responsive moisture-exchanger material. This process carries heat from CO2 responsive heat generating material to the temperature-responsive moisture-exchanger material. The amount and/or flow rate of this airflow may be controlled in order to optimise the transfer of heat.

The temperature-responsive moisture-exchanger material may also be heated from contact with the expiration airflow which will generally have a higher temperature than the surrounding atmosphere and parts of the respiratory humidify device.

At 940, the temperature of the temperature-responsive moisture-exchanger material increases above a critical temperature due to the transfer of heat from the CO2 heat generating material. This may also be helped by transfer of heat from the expiration airflow. Above this critical temperature, the temperature-responsive moisture-exchanger material changes state from hydrophilic to hydrophobic. That is, instead of absorbing moisture from a surrounding airflow, the temperature-responsive moisture-exchanger material releases moisture to the surrounding airflow.

At 945, the respiratory humidify device receives an inspiration airflow from a ventilator. This corresponds to air for the patient's in-breath and may be fully or partially conditioned at the ventilator and then delivered via an airflow pathway such as plastic tubing to the device. Conditioning may include humidification of captured surrounding air or stored gaseous compounds such as Oxygen. Conditioning may also include heating of the inspiration airflow. Travel along the tubing may cause the inspiration airflow to lose humidity and/or heat, which the respiratory humidifying device is able to compensate for. Alternatively, the device may provide all the conditioning required for the inspiration airflow.

At 950, the inspiration airflow is directed to contact the temperature-responsive moisture-exchanger material. This may be implemented passively by suitable configuration of an airflow pathway from a port receiving the inspiration airflow to the temperature-responsive moisture exchanger material. In some configurations, this airflow pathway may first direct the inspiratory airflow to contact the CO2 responsive heat generating material, before flowing on to contact the temperature-responsive moisture exchanger material. Again, this may be implemented using a passive arrangement of airflow pathway components such as ports and chambers holding the two materials. Alternatively, an active pathway may be used, for example by employing valves controlled to allow the inspiration airflow to contact one or both materials only during prescribed time periods. These time periods may be synchronised with the patient's breathing cycle.

At 955, the temperature-responsive moisture exchanger material releases moisture into the inspiration airflow, thereby humidifying the inspiration airflow. In some embodiments, the inspiration airflow may also be directed to contact the CO2-responsive heat generating material which, due to its increased temperature from reacting to the CO2 in the expiration airflow, will transfer heat to the inspiration airflow. In this way, the inspiration airflow can be both heated and humidified.

At 960, the humidified (and in some examples heated) inspiration airflow is directed to the patient. This may be via the same airflow pathway in which the expiration airflow is received from the patient.

In this example, a passive method of operation of a respiratory humidifying device may be implemented. This is simple to apply, cost-effective and may be psychologically soothing for the patient. In some examples, the method and device may be us by the patient alone, outside of the context of an operating theatre, or a high dependency unit in a hospital. In other examples, some active control features may be added to achieve better performance at the cost of higher price or increased complexity. For example, valves may be controlled to ensure the inspiration and expiration airflows are directed to the materials at optimal times during a patient's breathing cycle. Similarly, some additional heating may be provided to the inspiratory airflow and/or the temperature-responsive moisture exchanger material in order to supplement that provided by the CO2 responsive heat generating material.

FIG. 11 and FIG. 12 illustrate experimental results from testing a respiratory humidifying device on the human respiratory system with a facemask containing the PNIPAM-cotton material and a Sodalime layer. Measurement was carried out without the respiratory humidifying device operating to measure the temperature inside the mask under normal breathing conditions.

The experiment was performed in two stages: The first measures the temperature produced by the exothermic reaction of Soda-lime (Spherasorb) with CO2 gas from human breathing. In the control stage, the temperature produced by regular breathing (control) under the same conditions is compared with the experiment's first phase. Each stage lasts for about five minutes.

The first experiment started to measure the heat generated by the exothermic reaction when Soda-lime reacts with CO2 gas exhaled from human breathing through the Soda-lime (Spherasorb) layer and defines an optimal location for obtaining the heat since the soda-lime exothermic reaction is zonal. However, the thermocouple sensor was placed in the center of the soda-lime in the initial process.

The respiratory humidifying device was connected to a conventional mask, and the system was measured to calculate the time needed to achieve a maximum polymer upper critical solution temperature (UCST). Finally, the volunteer was invited to wear the mask without the respiratory humidifying device but with a CPAP humidifier working at maximum humidification and breathing for five minutes.

The results from the first clinical trial show the measured temperature generated by the exothermic reaction between the exalted air and the Soda-lime layer stored inside it. FIG. 11 demonstrates that the exemplary temperature inside the soda-lime layer can be up to 49.7 degrees Celsius in five minutes and stay constant above the polymer's upper critical solution temperature (UCST) of 39° C. In contrast, without using the respiratory humidifying device, the temperature inside the system will stay at 32 degrees Celsius for more than five minutes of the control trial, which is lower than polymer lower critical solution temperature (LCST) of between 25° C. and 33° C.

The second experiment aimed to determine and measure the difference in temperature between air moving from the patient to the heat-exchanging element (Tin) and the temperature of the air coming out from the element (Tout). The experiment had three stages, each with a different element integrated with the heat and moisture exchange system.

The elements involved in the experimental trial are Aluminum foils (Al), which is Aluminum prepared in light and thin metal with a thickness of less than 0.2Â angstrom, corrugated paper (C6H10O5) coated with Calcium chloride salt (CaCl2). The treated and corrugated paper, which is often coated with Calcium chloride salt to enhance the element hygroscopicity, is prepared from light and thin metal with a thickness of less than 0.4Â angstrom; and the third under investigation was the Soda-lime (Spherasorb) layer integrated into the respiratory humidifying device.

The results from the three conducted experiments are shown in the graph of FIG. 12. It illustrates the various heat-exchanging abilities of the three materials under investigation and comparison. The results showed different heat-exchanging abilities for the materials under examination. Aluminum foils performed poorly in breathing heat exchanging to retain one degree Celsius only. In comparison, Corrugated paper showed a somewhat better result with three and a half degrees whilst Soda lime crystals showed five degrees Celsius, demonstrating clearly the Sodalime layer's superiority.

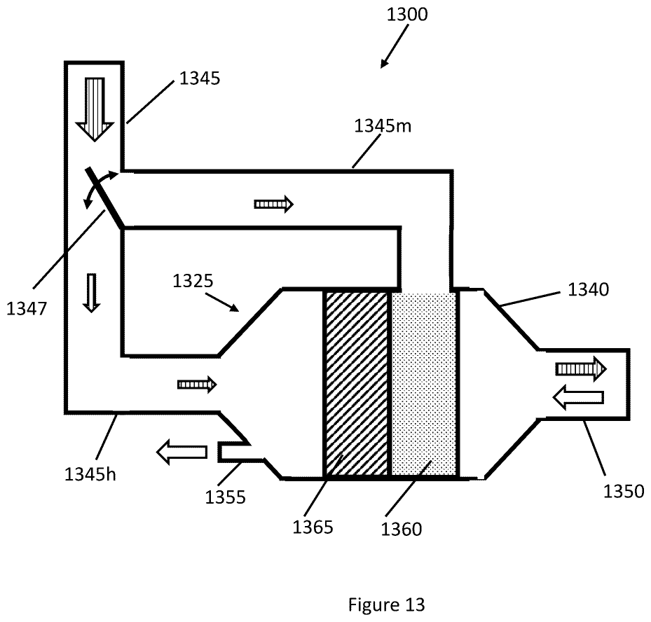

FIG. 13 shows a respiratory humidifying device according to some embodiments. The respiratory humidifying device 1300 is similar to the device 200 of FIG. 2, comprising a temperature-response moisture-exchanger material 1360 in contact with a CO2-responsive heat generating material 1365 within a housing 1340. A common port delivers humidified inspiratory airflow to a patient and receives expiratory airflow from the patient. In this example, expiratory airflow is vented through an expiratory port 1355 alternative pathways are possible.

The respiratory humidifying device 1300 comprises a bypass arrangement. Inspiratory airflow is received at an inspiratory port 1345 which splits into two limbs, a heating limb 1345h and a moisture limb 1345m. A valve or switch 1347 controls the proportion of inspiratory airflow that passes through each limb 1345h, 1345m. This is illustrated by the large arrow with shading being split into two smaller arrows with shading. The heating limb 1345h directs inspiratory airflow through the CO2-responsive heat generating material 1365 and the temperature-response moisture-exchanger material 1360. The moisture limb 1345m only directs inspiratory airflow through the temperature-response moisture-exchanger material 1360, and not through the CO2-responsive heat generating material 1365.

When the proportion of the inspiratory airflow that passes through the heating limb 1345h is reduced and the proportion which passes through the moisture limb 1345m is increased, the amount of heating of the inspiratory airflow delivered to the patient is reduced. Conversely, when the proportion of the inspiratory airflow that passes through the moisture limb 1345m is reduced and the proportion which passes through the heating limb 1345h is increased, the amount of heating of the inspiratory airflow delivered to the patient is increased. The amount of humidifying of the inspiratory airflow is largely unaffected.

This bypass arrangement may be configured to control the proportion of inspiratory air directed to contact the CO2-responsive heat generating material 1365 which in turn controls the overall level of heating of the inspiratory airflow delivered by the respiratory humidifying device 1300 to the patient. This may be controlled dependent on a characteristic of the inspiration airflow and/or the CO2-responsive heat generating material 1365. Examples of characteristics that may be used include: a temperature of the inspiration airflow (received or delivered); a relative humidity of the inspiration airflow (received or delivered); a temperature of the CO2-responsive heat generating material.

These characteristics may be determined by respective sensors and used by a controller to adjust the valve 1347. This may be implemented using an algorithm employing one or more of the characteristics which could be implemented by a microcontroller. In another example, the bypass arrangement may be implemented by using a bi-metallic strip within the inspiratory port 1345. As the temperature of the received inspiratory air increases, the bi-metallic strip may deform to reduce the proportion of inspiratory airflow directed through the heating limb 1345h. A bi-metallic strip located within the common port 1350 may alternatively be used to adjust the proportion the proportion of inspiratory airflow directed through the heating limb 1345h depending on the temperature of the airflows in the common port. This may be used to control the valve 1347 using a mechanical linkage. An advantage of using a bi-metallic strip or other mechanical control mechanism is that electrical power does not need to be supplied to the respiratory humidifying device, reducing cost and complexity.

Some embodiments provide a number of advantages including passive humidifying and/or heating of inspired air. This may be achieved without the need for external power supply or batteries, simplifying the process and associated respiratory humidification device. The device is also cheaper and smaller, and may even be made disposable. Embodiments may also reduce the number of components required, thereby reducing preparation time for use, as well as hazards such as infection or even electrocution.

Whilst some embodiments have been described with respect to a particular application, for example medical mechanical ventilation, many other applications are possible such as humidifying tank supplied gases for scuba-diving or humidifying atmospherically derived air for more comfortable breathing during a flight, visiting low humidity locations or other applications. Any and all references to publications or other documents, including but not limited to, patents, patent applications, articles, webpages, books, etc., presented anywhere in the present application, are herein incorporated by reference in their entirety.

As noted elsewhere, the disclosed inventive embodiments have been described for illustrative purposes only and are not limiting. Other embodiments are possible and are covered by the disclosure, which will be apparent from the teachings contained herein. Thus, the breadth and scope of the disclosure should not be limited by any of the above-described embodiments but should be defined only in accordance with claims supported by the present disclosure and their equivalents. Moreover, embodiments of the subject disclosure may include methods, systems and apparatuses/devices which may further include any and all elements from any other disclosed methods, systems, and devices, including any and all elements corresponding to binding event determinative systems, devices and methods. In other words, elements from one or another disclosed embodiments may be interchangeable with elements from other disclosed embodiments. In addition, one or more features/elements of disclosed embodiments may be removed and still result in patentable subject matter (and thus, resulting in yet more embodiments of the subject disclosure). Also, some embodiments correspond to systems, devices and methods which specifically lack one and/or another element, structure, and/or steps (as applicable), as compared to teachings of the prior art, and therefore, represent patentable subject matter and are distinguishable therefrom (i.e., claims directed to such embodiments may contain one or more negative limitations to note the lack of one or more features prior art teachings).

Various inventive concepts disclosed herein may be embodied as one or more methods (as so noted). The acts performed as part of the method may be ordered in any suitable way. Accordingly, embodiments may be constructed in which acts are performed in an order different than illustrated, which may include performing some acts simultaneously, even though shown as sequential acts in illustrative embodiments.

1. A respiratory humidifying device for humidifying inspiration airflow, the device comprising:
a temperature-responsive moisture-exchanger material configured to be in contact with the inspiration airflow and an expiration airflow;
a CO2-responsive heat generating material configured to be in contact with at least the expiration airflow;
wherein the CO2-responsive heat generating material is in thermal communication with the temperature-responsive moisture-exchanger material.

2. The device of claim 1, wherein the CO2-responsive heat generating material is configured to generate heat upon contact with the expiration airflow and to transfer at least some of the heat to the temperature-responsive moisture-exchanger material;
wherein the temperature-responsive moisture-exchanger material is configured to change temperate from below a critical solution temperature to above the critical solution temperature in response to the transfer of the heat from the CO2-responsive heat generating material; and
wherein the temperature-responsive moisture-exchanger material is configured to release moisture above the critical solution temperature.

3. The device of claim 2, wherein the critical solution temperature is between 25° C. and 39° C.

4. The device of claim 2, wherein the temperature-responsive moisture-exchanger material has a second critical solution temperature below which the temperature-responsive moisture-exchanger material is configured to absorb moisture.

5. The device of claim 4, wherein the temperature-responsive moisture-exchanger material is configured to have a first temperature range between the first and second critical temperatures and the CO2-responsive heat generating material is configured to change temperature by a second temperature range responsive to being in contact with the expiration airflow, the second temperature range overlapping the first temperature range.

6. The device of claim 5, wherein the first temperature range is 3-6 C and the second temperature range is 3-10 C.

7. The device of claim 1, wherein the CO2-responsive heat generating material configured to be in contact with the inspiration airflow.

8. The device of claim 7, comprising a by-pass arrangement configured to direct only a part of the inspiration airflow to contact the CO2-responsive heat generating material.

9. The device of claim 8, wherein a proportion of the inspiration airflow directed to contact the CO2-responsive heat generating material is dependent on a characteristic of the inspiration airflow and/or the CO2-responsive heat generating material.

10. The device of claim 9, wherein the characteristic comprises one or more of the following: a temperature of the inspiration airflow; a relative humidity of the inspiration airflow; a temperature of the CO2-responsive heat-generating material.

11. The device of claim 8, wherein the bypass arrangement comprises a bi-metallic strip.

12. The device of claim 1, further comprising:
an inspiration port to receive the inspiration airflow;
a common port to supply humidified inspiration airflow from and to receive the expiration airflow; and
an expiration port to vent the expiration airflow.

13. The device of claim 1, wherein the CO2-responsive heat-generating material comprises one or more of the following: Soda-lime; Sodasorb; Spherasorb.

14. The device of claim 1, wherein the temperature-responsive moisture-exchanger material comprises one or more of the following: PNIPAM; poly(N,Ndiethylacrylamide) (PDEAM).

15. The device of claim 1, wherein the temperature-responsive moisture-exchanger material is configured to be in physical contact with the CO2-responsive heat generating material.

16. The device of claim 1, wherein the temperature-responsive moisture-exchanger material and the CO2-responsive heat generating material are provided on a fabric substrate.

17. A replaceable cartridge for a respiratory apparatus comprising the respiratory humidifying device of claim 1.

18. A respiratory apparatus comprising the respiratory humidifying device of claim 1.

19. The respiratory apparatus of claim 18, further comprising a ventilating apparatus to generate the inspiration airflow.

20. A method of humidifying inspiration airflow, the method comprising:
directing an expiration airflow to contact a temperature-responsive moisture-exchanger material and a CO2-responsive heat generating material in thermal communication with the temperature-responsive moisture-exchanger material;
directing an inspiration airflow to contact the temperature-responsive moisture-exchanger material.

21. The method of claim 20, further comprising:
the CO2-responsive heat generating material reacting with CO2 in the expiration airflow to generate heat to transfer to the temperature-responsive moisture-exchanger material;
the temperature-responsive moisture-exchanger material increasing temperate from below a critical solution temperature to above the critical solution temperature in response to receiving the heat transferred from the CO2-responsive heat generating material; and
wherein the temperature-responsive moisture-exchanger material is configured to release moisture above the critical solution temperature.
